(19) United States
(12) Patent Application Publication
(10) Pub. No.: US 2019/0134271 A1
(43) Pub. Date: May 9, 2019

(54) YARN FOR CELL CULTURE SCAFFOLD, AND FABRIC INCLUDING THE SAME FOR CELL CULTURE SCAFFOLD

(71) Applicant: AMOLIFESCIENCE CO., LTD., Seoul (KR)

(72) Inventors: In Yong SEO, Seoul (KR); Seon Ho JANG, Seoul (KR); Song Hee KOO, Gimpo-si (KR); Chan KIM, Gwangju (KR); Seoung Hoon LEE, Paju-si (KR)

(21) Appl. No.: 16/304,321

(22) PCT Filed: May 24, 2017
(86) PCT No.: PCT/KR2017/005423

§ 371 Date: Nov. 26, 2018

(30) Foreign Application Priority Data
May 24, 2016 (KR) 10-2016-0063692

(51) Int. Cl. A61L 27/38; D02G 1/02; C12N 5/0735; C12N 5/00; C12N 5/079; C12N 5/077

(52) U.S. Cl. CPC A61L 27/3834; D02G 1/02; C12N 5/0606; C12N 5/0654; C12N 5/0618; C12N 5/0658; C12N 5/0653; C12N 5/0062

(57) ABSTRACT

Provided is yarn for a cell culture scaffold. The yarn includes ply-twisted fiber strands, and to prevent density-dependent inhibition of cultured cells and increase a cell-contacting specific surface area, at least a part of the plurality of twisted fiber strands are untwisted such that an open space is formed between the fibers. A cell proliferation rate and cell viability may be increased by creating microenvironments suitable for migration, proliferation and differentiation of the cultured cells using the yarn. A large quantity of cells may be simultaneously cultured by creating a cell proliferation space as large as possible in a scaffold space having a limited cell proliferation space, and cell proliferation may be steadily maintained by preventing the inhibition of cell proliferation due to intercellular contact. The cells cultured may be cultured to have a shape/structure suitable for application to an in vitro experiment model or implantation into an animal body.

### TECHNICAL FIELD

The present invention relates to yarn for a cell culture scaffold, and more particularly, to yarn for a cell culture scaffold which is improved in cell viability by creating microenvironments suitable for adhesion, migration, proliferation and differentiation of cultured cells, allows cells to be three-dimensionally proliferated, prevents density-dependent inhibition by contact between cells proliferated in a limited space according to cell proliferation and increases a specific surface area with which cells can contact, ply yarn including the same, and a fabric including the same.

### BACKGROUND ART

Recently, according to expansion of the use of cultured cells in disease treatment, interest and research on cell culture are increasing. Cell culture is a technique for collecting cells from a living organism and culturing the cells outside the living organism, and the cultured cells may be used in treatment of various diseases through differentiation into various types of tissue of a body, for example, the skin, organs, nerves, etc. to be implanted into the body, or implantation in an undifferentiated state to attain engraftment and differentiation at the same time.

A field associated with such cell culture is tissue engineering, which is an interdisciplinary study that applies existing scientific fields such as cytology, life science, engineering, medicine, etc., and thus novel fusion technology for understanding a relationship between the structure and function of living tissue, replacing damaged tissue or a damaged organ with normal tissue and regenerating the damaged tissue or organ has been studied.

Such fusion technology is continuously receiving great attention in a conventional cell culture field or a tissue engineering field using the same, and one of tasks which are being studied and developed is a study of a material and structure of a scaffold which can culture/differentiate cells and be implanted into human tissue while including the cells.

However, the scaffolds for a cell culture which have been developed until now have a structure similar to the body, but do not allow cells to be three-dimensionally cultured and not have high cell viability, and therefore, cells cultured thereby are not suitable for being used in an in vitro experiment model or as cells for implantation.

In addition, cells that are two- or three-dimensionally cultured in a limited space during cell proliferation may not be cultured to a desired level due to density-dependent inhibition of cell growth between adjacent cells.

Therefore, there is an urgent demand for development of a scaffold which can three-dimensionally culture cells to a desired level by increasing a specific surface area capable of culturing cells and also preventing density-dependent inhibition of cell growth, which may occur in cell proliferation.

### DISCLOSURE

### Technical Problem

The present invention is devised by taking the above-mentioned problems into account, and thus directed to providing yarn for a cell culture scaffold which is improved in cell proliferation rate and cell viability by creating microenvironments suitable for migration, proliferation and differentiation of cultured cells.

In addition, the present invention is also directed to providing yarn for a cell culture scaffold which is significantly increased in proliferation space for cells cultured in a limited region of the scaffold.

Further, the present invention is also directed to providing yarn for a cell culture scaffold which has an environment capable of continuously proliferating cells by preventing density-dependent inhibition of cell growth occurring due to intercellular contact.

Moreover, the present invention is also directed to providing a fabric for a cell culture scaffold, which can be widely applied in various types of products used in a cell culture or tissue engineering field, including a bioreactor, a cell culture container, an implantable kit, etc., using the yarn according to the present invention.

Furthermore, the present invention is also directed to providing an implant for tissue engineering by using a cell cluster three-dimensionally cultured to be suitable for implantation into a living organism using the fabric according to the present invention.

### Technical Solution

To solve the above-described problems, the present invention provides yarn for a cell culture scaffold, which includes a ply-twisted fiber strands, and has an open space between fibers by untwisting at least a part of the ply-twisted fiber strands to prevent density-dependent inhibition of cells to be cultured and increase a cell-contacting specific surface area.

According to an exemplary embodiment of the present invention, the fiber may be spun yarn, filament yarn or slitting yarn.

In addition, the fiber may include, as a fiber-forming component, any one or more non-biodegradable components selected from the group consisting of polystyrene (PS), polyethylene terephthalate (PET), polyethersulfone (PES), polyvinylidene fluoride (PVDF), polyacrylonitrile (PAN), polydimethylsiloxane (PDMS), a polyamide, a polyalkylene, a poly(alkylene oxide), a poly(amino acid), a poly(allylamine), polyphosphazene and a polyethyleneoxide-polypropyleneoxide block copolymer, or any one or more biodegradable components selected from the group consisting of polycaprolactone, polydioxanone, polyglycolic acid, poly(L-lactide) (PLLA), poly(DL-lactide-co-glycolide) (PLGA), polylactic acid and polyvinyl alcohol.

In addition, the yarn may have a fineness of 20 to 300 deniers, and the fiber may have a fineness of 0.1 to 30 deniers.

In addition, the slitting yarn may be a fiber web with a three-dimensional network structure cut to have a predetermined width. Here, the fiber web may have a basis weight of 0.1 to 100 g/m², and a width of 0.1 to 30 mm.

In addition, the fiber may further include a physiologically active component inducing any one or more of adhesion, migration, growth, proliferation and differentiation of cells on an outer surface. Here, the physiologically active component may include any one or more among any one or more compounds selected from the group consisting of a monoamine, an amino acid, a peptide, a saccharide, a lipid, a protein, a glucoprotein, a glucolipid, a proteoglycan, a mucopolysaccharide and a nucleic acid, and a cell.

In addition, the yarn for a cell culture scaffold may be used for a scaffold to culture any one or more types of stem cells selected from the group consisting of totipotent stem cells, pluripotent stem cells, multipotent stem cells, oligopotent stem cells and single stem cells, and one or more types of differentiated cells selected from the group consisting of hematopoietic stem cells, liver cells, fiber cells, epithelial cells, mesothelial cells, endothelial cells, muscle cells, nerve cells, immune cells, adipose cells, cartilage cells, bone cells, blood cells and skin cells.

In addition, the present invention provides a fabric for a cell culture scaffold, which includes the yarn according to the present invention.

In addition, the present invention provides an implant for tissue engineering, which includes the fabric according to the present invention; and cells cultured while in contact with yarn for a cell culture scaffold included in the fabric.

According to an exemplary embodiment of the present invention, cells are provided in contact with fibers spaced apart from each other in the yarn for a cell culture scaffold, and intercellular contact may be prevented by arranging the fibers between adjacent cells among the cells.

In addition, the cells may include any one or more types of stem cells selected from the group consisting of totipotent stem cells, pluripotent stem cells, multipotent stem cells, oligopotent stem cells and single stem cells, and one or more types of differentiated cells selected from the group consisting of hematopoietic stem cells, liver cells, fiber cells, epithelial cells, mesothelial cells, endothelial cells, muscle cells, nerve cells, immune cells, adipose cells, cartilage cells, bone cells, blood cells and skin cells.

Hereinafter, terms used in the present invention will be described.

The term “extracellular matrix (ECM)” used herein is a substrate which surrounds the outside of a cell, occupies a space between cells, and has a network structure usually consisting of proteins and polysaccharides.

The “motif” used herein is a peptide comprising an amino acid sequence, which can structurally/functionally interact with a receptor included in a protein, a glucoprotein, etc. in the ECM playing a critical role in cell adhesion, migration, differentiation, etc. to pass through a surface of a cell membrane or a membrane, and is isolated from a cell or artificially produced using a gene cloning technique.

The term “three-dimensional cell cluster” used herein refers to a group of cells which are three-dimensionally collected.

### Advantageous Effects

According to the present invention, microenvironments suitable for migration, proliferation and differentiation of cells cultured can be increased by yarn, thereby improving a cell proliferation rate and cell viability.

In addition, a large quantity of cells can be simultaneously cultured by creating a cell proliferation space as large as possible in a scaffold space having a limited cell proliferation space, and cell proliferation can be steadily maintained by preventing the inhibition of cell proliferation due to intercellular contact.

Further, as a surface area in which cells can be cultured is increased, a cell proliferation rate increases, and as a distance between proliferated cell clusters is larger, proliferation between cell clusters is not hindered, more improved culturability can be exhibited.

Furthermore, an increased distance between cell clusters can lead to increased freedom of choice of a migration pathway during migration, thereby further increasing a migration rate and a proliferation rate, which is advantageous to cell culture.

Therefore, the cultured cells can be three-dimensionally cultured in a shape/structure more suitable for being applied to an in vitro experimental model or being implanted in the body of an animal, and can be widely applied in various products used in a cell culture or tissue engineering field, for example, a bioreactor, a cell culture container, a kit for implantation into a body, etc.

### DESCRIPTION OF DRAWINGS

FIG. 1 includes a perspective view and a partially-enlarged view of yarn according to an exemplary embodiment of the present invention.

FIG. 2 is a perspective view of the yarn according to the exemplary embodiment of the present invention.

FIGS. 3A and 3B show examples of slitting yarn included in an exemplary embodiment of the present invention, in which FIG. 3A is an enlarged view showing a state of a fiber web before being produced into slitting yarn, and FIG. 3B is an enlarged view showing a state of a fiber web after being produced into slitting yarn.

FIG. 4 is an exploded perspective view of the yarn according to the exemplary embodiment of the present invention, showing yarn produced by twisting slitting yarns provided as fibers.

FIG. 5 is an SEM image showing that cells are cultured while being surrounded by fibers in the yarn according to the exemplary embodiment of the present invention.

FIG. 6 is an SEM image showing that a cell cluster is cultured on the surface of a fiber in the yarn according to an exemplary embodiment of the present invention.

FIG. 7 is an image of a 1.7M wide nanofiber web for producing slitting yarn included in an exemplary embodiment of the present invention (a) and a scanning electron microscope image of the nanofiber web (b).

FIG. 8 shows a set of images showing an intermediate step for producing slitting yarn according to an exemplary embodiment of the present invention, in which (a) is an image of slitting yarn produced by primary slitting to a width of 50 mm, (b) is an image illustrating a process of precisely slitting the yarn obtained through the primary slitting to a width of 1.5 mm, and (c) is an image illustrating a process of winding the slitting yarn with a width of 1.5 mm, produced as described in (b).

FIG. 9A is an SEM image showing yarn before being partially untwisted in a process of producing yarn for a cell scaffold according to an exemplary embodiment of the present invention.

FIG. 9B is an SEM image showing yarn for a cell scaffold according to an exemplary embodiment of the present invention, which is produced by partially untwisting the yarn according to FIG. 9A.

FIG. 10 is an image of a cone wound with slitting yarns after braiding and twisting (a), and an electron microscope image of the twisted slitting yarns (b).

### MODE OF INVENTION

Hereinafter, embodiments of the present invention will be described in detail with reference to the accompanying drawings so that those of ordinary skill in the art can easily carry out the present invention. The present invention may be implemented in a variety of different forms, and is not limited to the embodiments described herein. For clear explanation of the present invention in the drawings, parts that are not related to the description are omitted, and the same numerals denote the same or like components throughout the specification.

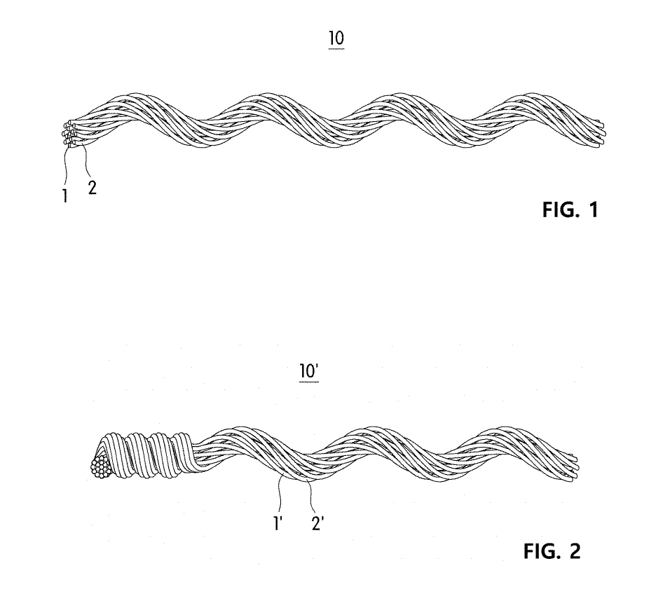

As shown in FIG. 1, yarn 10 for a cell culture scaffold according to an exemplary embodiment of the present invention includes a ply-twisted fiber strands 1 and 2, and an open space between fibers by untwisting a part or all of the ply-twisted fiber strands.

When yarn is realized without an open space between fibers by twisting a plurality of fiber strands, cells adhered to the yarn may not enter into the yarn, and highly tend to be two- or three-dimensionally proliferated along the outer surface. However, in the case of two-dimensional proliferation, an area in which the cells can be cultured is limited to the outer surface of a scaffold for cell culture, and it may be difficult to proliferate a desired amount of cells with a limited volume of a scaffold. Such cells have more influence on cells proliferating in an elongated shape, such as muscle cells, nerve cells, fibroblasts, etc., or stem cells, and an increase in volume of the scaffold to solve such problem may not be a preferable method since it requires a change of a cell culture container or a culture instrument.

However, when intercellular contact is increased in cell proliferation, a cell division rate may slow down and stop at any moment, which is called density-dependent inhibition of cell growth. All normal cells excluding abnormal cells such as cancer cells have the above-mentioned characteristic, and when the proliferation of cells cultured in a limited space continues to reach more than a predetermined level of the density of the proliferated cells, due to excessive intercellular contact, a cell proliferation rate may slow down and thus the proliferation may stop. When this phenomenon occurs under an in vitro environment for intentionally culturing cells, it may have a problem in that cells cannot be cultured at a desired amount or in a desired shape. As a result of continuous study to solve this problem, it was found that the surface area of the yarn capable of contacting cells may be significantly increased and improved intercellular contact may be indirectly prevented by significantly increasing by adjusting a volume of the yarn for a cell scaffold with a limited length, and intercellular contact may be directly prevented by disposing a fiber among adjacent cells, and thus the present invention was completed.

Referring to FIG. 1, a fiber strands 1 and 2 are twisted in any one direction, but untwisted to form a space between the fibers 1 and 2, which are spaced apart. In this case, a volume of the yarn 10 is increased as large as the volume of the formed space, and therefore the surface area of the outer surface of the yarn 10 is increased. In addition, as a space is formed in the yarn 10, cells may be proliferated on a fiber located on the outer surface of the yarn 10 and also on a fiber located in an inner space after being migrated, and therefore the surface area of the scaffold in which cells can be cultured may further increase. In addition, in this case, the cells proliferated on the outer surface of the yarn 10 and the cells proliferated in the yarn may not have density-dependent inhibition of cell growth by directly preventing intercellular contact due to a fiber located between the cells. Moreover, cells are cultured outside and inside of the yarn 10, not two-dimensionally cultured along the outer surface of the yarn 10, which, ultimately, may be more preferable for obtaining a three-dimensionally-cultured cell cluster. However, when untwisting is excessively performed, the formed space is so large that small-sized cells may be released from the scaffold, and therefore the fibers should be untwisted to an extent that ensures a suitable space. In addition, it is preferable that the number of fiber strands is increased and spaced apart to ensure a surface area preferable for cell growth.

Meanwhile, a space formed between separated fibers may be formed in the entire region of the yarn for a cell scaffold as shown in FIG. 1, or an open space between fibers may be formed by untwisting only a part (A) of twisted yarn 10′ as shown in FIG. 2.

A degree of untwisting the twisted yarn 10 or 10′ may be determined by considering a type and a size of cells to be cultured, and a shape and a size of a cell cluster. However, when untwisting is excessively performed, bulkiness of the yarn may increase, but mechanical strength of the yarn is degraded. If there is an external physical force applied to a cell culture environment, for example, cells are cultured in a consistently-circulated culture medium, not in a culture medium in a stationary state, yarn excessively increased in bulkiness due to fluidity of the cell culture medium may not stably support the cells, and the culture cells may be detached from a scaffold. Therefore, for example, the twisted yarn, that is, ply yarn, may have a twist number of 100 to 5000 T/m, and an untwist rate represented by Mathematical Expression 1 to represent a degree of untwisting such yarn may be 10 to 60%.

Untwist rate (%)=(length (m) of yarn after untwisting−length (m) of ply yarn)×100/length (m) of ply yarn [Mathematical Expression 1]

The fineness of the yarn may be determined by considering a type and a size of cells to be cultured, and may be preferably 20 to 300 deniers. If the fineness is less than 20 deniers, due to a decrease in specific surface area to which cells are adhered, it can be difficult to produce a cell cluster to a desired level, and weavability may be degraded when a fabric is produced with yarn. In addition, when the fineness is more than 300 deniers, due to an excessive diameter of the scaffold, loaded cells may be grown intermittently, rather than being proliferated to form a three-dimensional group, and it may be difficult to obtain a cell cluster having uniform size and shape.

In addition, the yarn may consist of a plurality of fiber strands, and the number of fibers included in the yarn may be suitably changed to meet the type and size of cells to be cultured, and the shape and size of a cell cluster, and thus the present invention is not particularly limited thereto.

The fiber 1, 1′, 2 or 2′ included in the yarn may be spun yarn, filament yarn or slitting yarn.

When the fiber is spun yarn or filament yarn, the fineness of the fiber may be 0.1 to 30 deniers. However, the present invention is not limited thereto, and the fineness of the fiber may be changed to be suitable for the type and size of cells to be cultured, and the shape and size of a cell cluster.

In addition, the spun yarn may be produced from raw cotton by a known method. In addition, the filament yarn may be produced by spinning according to a known method, and the spinning may be performed by a known spinning method such as chemical spinning or electrospinning.

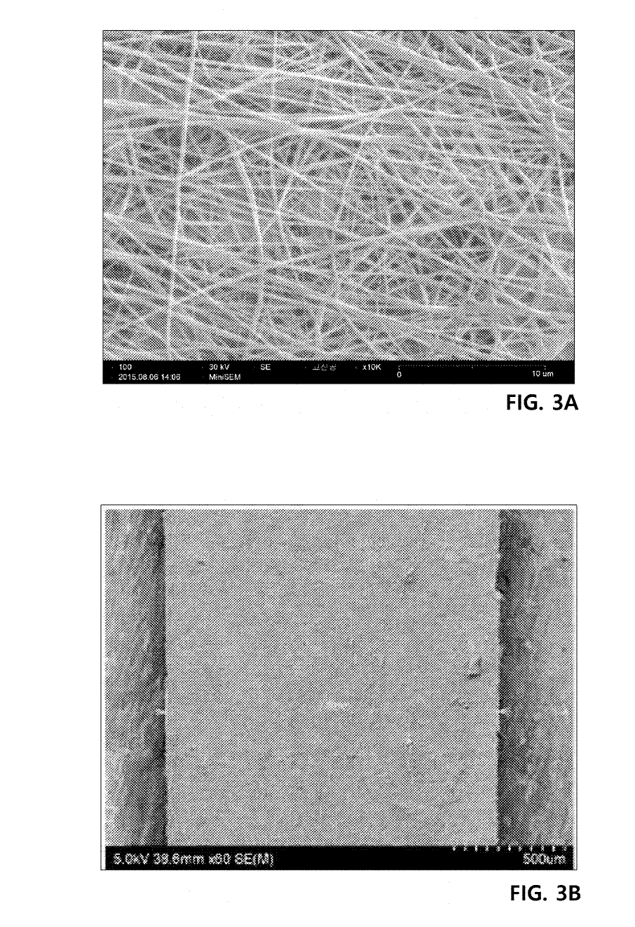

In addition, the slitting yarn may be produced by cutting a sheet-type fiber assembly or a fabric to have a predetermined width. Preferably, the slitting yarn may be fiber produced by cutting a sheet-type fiber web having a three-dimensional network structure to have a predetermined width. Here, the fiber web may be compressed with a constant pressure to improve the ease of a slitting process, and increase the strength of the slitting yarn. For example, FIG. 3A shows a sheet-type nanofiber web having a three-dimensional network structure, which may be compressed and cut to a predetermined width, thereby producing slitting yarn as shown in FIG. 3B. The slitting yarn implemented by the fiber web having a three-dimensional network structure has fine fibers such as nanofibers constituting the fiber web, and therefore cells may be more tightly adhered to the yarn. In addition, when the size of the cells to be cultured is small, a fine space in the fiber web may provide another culture space in which cells will be cultured. Moreover, since a cell culture solution can pass through the fiber web, the yarn itself, which is produced through twisting, and partially or entirely untwisting, has permeability with respect to a cell culture solution, and therefore cells may be more stably cultured with high efficiency.

The slitting yarn may be a fiber produced by cutting a fiber web having a basis weight of 0.1 to 100 g/m², preferably, 0.1 to 50 g/m², and more preferably, 0.1 to 20 g/m² to have a width of 0.1 to 30 mm. If the fiber web is slit to have a width of less than 0.1 mm, the fiber web is not easily cut, and may be easily broken due to tension and torque applied during twisting, and partial or entire untwisting. In addition, when the fiber web is slit to have a width of more than 30 mm, an irregular twist may be formed during twisting. In addition, when the basis weight of the slitting yarn is less than 0.1 g/m², the mechanical strength of the slitting yarn is degraded, and thus cells may not be stably cultured, and when a fabric is produced from slitting yarn, weavability may be degraded. In addition, when basis weight of the slitting yarn is more than 100 g/m², due to heavy compression of the nanofiber web, characteristics of the nanofiber web as a scaffold for cell culture are degraded, and thus the tendency of cells to two-dimensionally grow only along an outer surface without migration into the nanofiber web may be more increased.

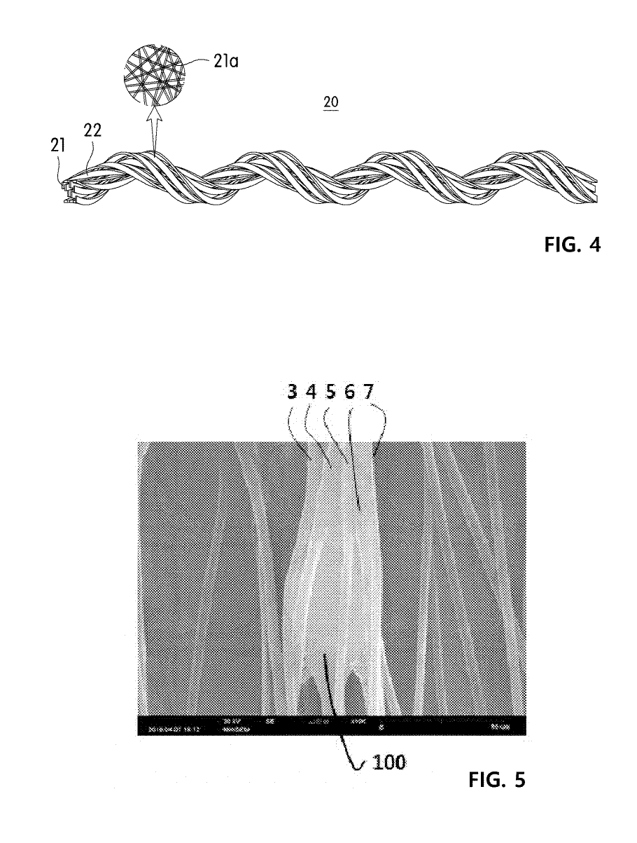

In the case of the above-described slitting yarn, as shown in FIG. 4, first slitting yarn 21 and second slitting yarn 22 are braided and twisted, and then totally untwisted, thereby realizing yarn 20 for a cell culture scaffold including an open space between slitting yarns 21 and 22.

The above-described fibers 1, 1′, 2, 2′, 21 and 22 may be produced of a known fiber-forming component capable of being produced in a fiber, and may be produced by selecting a suitable material depending on the type of a fiber. Since a material may vary depending on a specific purpose, for example, requirement of a decomposition property, the present invention is not particularly limited thereto.

The fiber-forming component may include a cellulose component such as cotton or hemp, a protein component such as wool or silk, or a natural fiber component such as a mineral component. In addition, the fiber-forming component may be a known artificial fiber component.

Meanwhile, the fiber-forming component may include any one or more non-biodegradable components selected from the group consisting of polystyrene (PS), polyethylene terephthalate (PET), polyethersulfone (PES), polyvinylidene fluoride (PVDF), polyacrylonitrile (PAN), polydimethylsiloxane (PDMS), a polyamide, a polyalkylene, a poly(alkylene oxide), a poly(amino acid), a poly(allylamine), polyphosphazene and a polyethyleneoxide-polypropyleneoxide block copolymer, or any one or more biodegradable components selected from the group consisting of polycaprolactone, polydioxanone, polyglycolic acid, poly(L-lactide) (PLLA), poly(DL-lactide-co-glycolide) (PLGA), polylactic acid and polyvinyl alcohol depending on purpose.

In addition, the above-described fibers may further include a functional material, in addition to the fiber-forming component. As an example of the functional material, the fiber may further include a physiologically active component inducing any one or more of cell adhesion, migration, growth, proliferation and differentiation. The physiologically active component may include any one or more among any one or more compounds selected from the group consisting of a monoamine, an amino acid, a peptide, a saccharide, a lipid, a protein, a glucoprotein, a glucolipid, a proteoglycan, a mucopolysaccharide and a nucleic acid, and a cell. The materials may be, specifically, present in the ECM.

However, the physiologically active component may include a motif. The motif may be a natural or recombinant peptide comprising a predetermined amino acid sequence included in any one or more selected from proteins, glucoproteins and proteoglycans included in a growth factor or the ECM. Specifically, the motif may include a predetermined amino acid sequence included in any one or more growth factors (GFs) selected from the group consisting of adrenomedullin, angiopoietin, a bone morphogenetic protein (BMP), a brain-derived neurotrophic factor (BDNF), an epithelial growth factor (EGF), erythropoietin, a fibroblast growth factor, a glial cell line-derived neurotrophic factor (GDNF), a granulocyte colony-stimulating factor (G-CSF), a granulocyte macrophage colony-stimulating factor (GM-CSF), growth differentiation factor-9 (GDF9), a hepatocytic growth factor (HGF), a hepatoma-derived growth factor (HDGF), an insulin-like growth factor (IGF), a keratinocyte growth factor (KGF), a migration-stimulating factor (MSF), myostatin (GDF-8), a nerve growth factor (NGF), a platelet-derived growth factor (PDGF), thrombopoietin (TPO), a T-cell growth factor (TCGF), neuropilin, transforming growth factor-α (TGF-α), transforming growth factor-β (TGF-β), tumor necrosis factor-α (TNF-α), a vascular endothelial growth factor (VEGF), IL-1, IL-2, IL-3, IL-4, IL-5, IL-6 and IL-7. Alternatively, the motif may include a predetermined amino acid sequence included in any one or more selected from the group consisting of hyaluronic acid, heparin sulfate, chondroitin sulfate, dermatan sulfate, keratan sulfate, alginate, fibrin, fibrinogen, collagen, elastin, fibronectin, bitronectin, carderine and laminin in the ECM. In addition, the motif may include both of a predetermined amino acid sequence included in the growth factor and a predetermined amino acid sequence included in the ECM. More preferably, the motif may include one or more selected from the group consisting of proteins comprising amino acid sequences of SEQ. ID. NO: 8 to SEQ. ID. NO: 28 and one or more selected from the group consisting of proteins in which at least two of the proteins are fused, but the present invention is not limited thereto.

Meanwhile, the motif may be integrated with the above-described adhesive component by a covalent bond. For example, when the adhesive component is a protein, the motif may be covalently bonded to the N-terminus and/or the C-terminus of a polypeptide directly or via a heterologous peptide or polypeptide, and in this case, the physiologically active component may be more tightly adhered to a scaffold fiber, and detachment of the physiologically active component during cell culture may be minimized.

In addition, as the physiologically active component, a known mussel protein or a specific domain or motif of a mussel protein may be included to improve cell adhesion.

The physiologically active component may be included while being fixed to the surface of a fiber, and as an example, the component may be included on the surface of a fiber by a coating process. In addition, the physiologically active component may be mixed with a fiber-forming component in a spinning solution for producing a fiber, and provided from a step of producing a fiber. In this case, it is advantageous that the physiologically active component may be easily provided to the outer surface of the produced fiber without a separate coating process or an adhesive component.

Meanwhile, the present invention provides a fabric for cell culture using the above-described yarn according to the present invention or yarn braided therewith.

The fabric may be any one of a woven fabric, a knitted fabric and a non-woven fabric, and the type of the fabric may vary depending on purpose. The woven fabric, knitted fabric and non-woven fabric may be produced by known corresponding methods. For example, the woven fabric may be a twill fabric produced by diagonally weaving the above-described yarn or cord braided therewith as any one or more of a warp and a weft. In addition, for example, the knitted fabric may be a flat knit fabric weft-knitted by putting the above-described yarn or cord braided therewith into a flat-knitting machine. In addition, for example, the non-woven fabric may be produced by adding an adhesive component to short-cut yarn formed by cutting the above-described yarn or yarn braided therewith to a predetermined fiber length and then applying heat/pressure thereto.

In addition, the present invention may provide an implant for tissue engineering, which includes cells cultured after cells to be cultured are implanted into the above-described fabric. Here, in a region including the outer surface of the yarn and an open space between untwisted fibers, the cells to be cultured may migrate to the space, and thus the culture cells may be located and cultured in the yarn. Here, the separated fibers may be located between adjacent cells among the cultured cells, and thereby, contact between the adjacent cells is directly prevented, and therefore such arrangement may be more advantageous for cell culture. Referring to FIG. 5, it can be seen that there is an opening space between a plurality of twisted fiber strands 3, 4, 5, 6 and 7, and a first cell 100 is cultured in the space while being in contact with the plurality of twisted fiber strands 3, 4, 5, 6 and 7. Here, even when a second cell (not shown) is cultured to be in contact with any one or more of the fibers 3, 4, 5, 6 and 7, contact between the first cell 100 and the second cell (not shown) may be prevented due to the adjacent fibers, and therefore density-dependent inhibition may be prevented.

In addition, the cells to be cultured may be, as shown in FIG. 6, different from FIG. 5, adhered to the outer surface of a first fiber 8, thereby culturing a first cell cluster A, and adhered to the outer surface of a second fiber 9 separated from the first fiber 8, thereby culturing a second cell cluster B. At this time, an increased distance between the first cell cluster (A) and the second cell cluster (B) leads cells included in each cluster to be increased in freedom of choice of a migration pathway during migration, and further increased in migration rate and proliferation rate, and thus is advantageous to cell culture.

Meanwhile, the cells may include one or more types of cells among any one or more stem cells selected from the group consisting of totipotent stem cells, pluripotent stem cells, multipotent stem cells, oligopotent stem cells and single stem cells, and differentiated cells selected from the group consisting of hematopoietic stem cells, liver cells, fiber cells, epithelial cells, mesothelial cells, endothelial cells, muscle cells, nerve cells, immune cells, adipose cells, cartilage cells, bone cells, blood cells and skin cells. As an example, the cells may be cells having a shape which is elongated in one direction, rather than a spherical shape, or cells having a high migration property. In addition, for the cells, for example, a type of stem cells tending to be cultured in a colony form may be more appropriate.

In addition, when a material of the fabric includes a fiber-forming component harmless to a human body, a cultured cells-adhered scaffold can be directly implanted into the human body, and therefore the culture cells can be more easily and stably engrafted into tissue.

Hereinafter, the present invention will be described in further detail with reference to examples. These examples are merely provided to exemplify the present invention, and it will be apparent to those of ordinary skill in the art that the scope of the present invention should not be construed as being limited to these examples.

### EXAMPLE 1

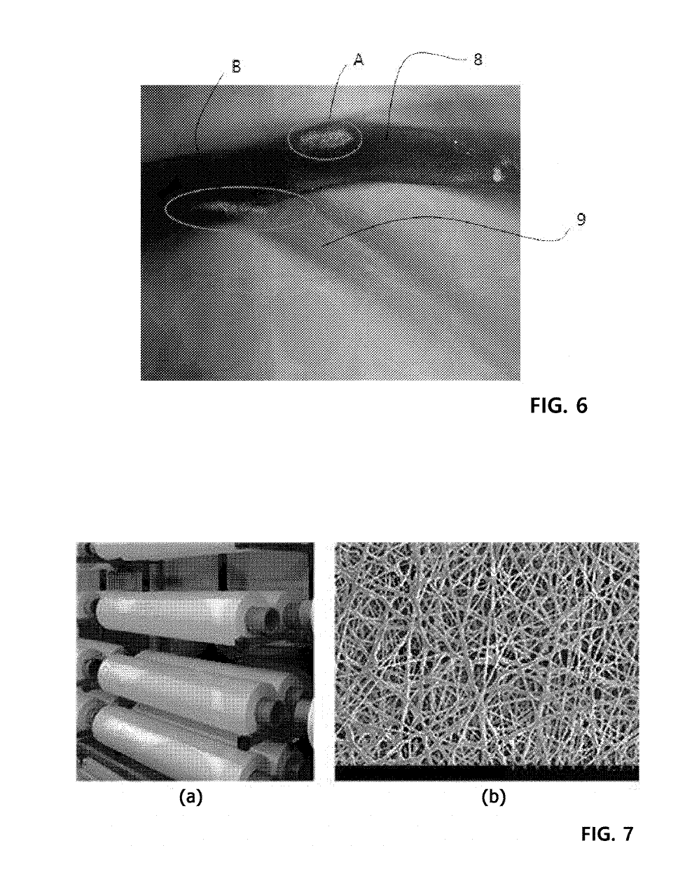

A spinning solution was prepared by dissolving PVDF as a fiber-forming component in DMAc/Acetone as a mixed solvent to have a concentration of 15 wt %. Electrospinning was performed using the prepared spinning solution and an electrospinning device under conditions of an applied voltage of 25 kV, a distance between a current collector and a spinning nozzle of 25 cm and a discharge amount of 0.05 ml/hole in an R.H. 65% environment at 30° C., thereby obtaining a roll of a nanofiber web having a width of 1.5 m, a basis weight of 5 g/m² and a length of 500 m. FIG. 7A is an image of a wound nanofiber web, and FIG. 7B is a scanning electron microscope image of the nanofiber web. As shown in FIG. 7B, an average diameter of a nanofiber forming the nanofiber web was approximately 230 nm.

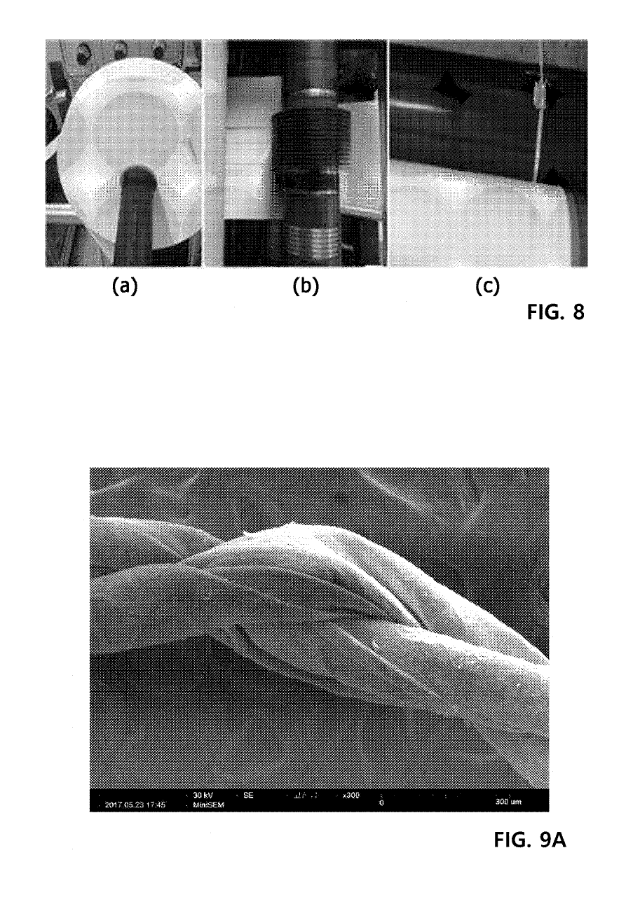

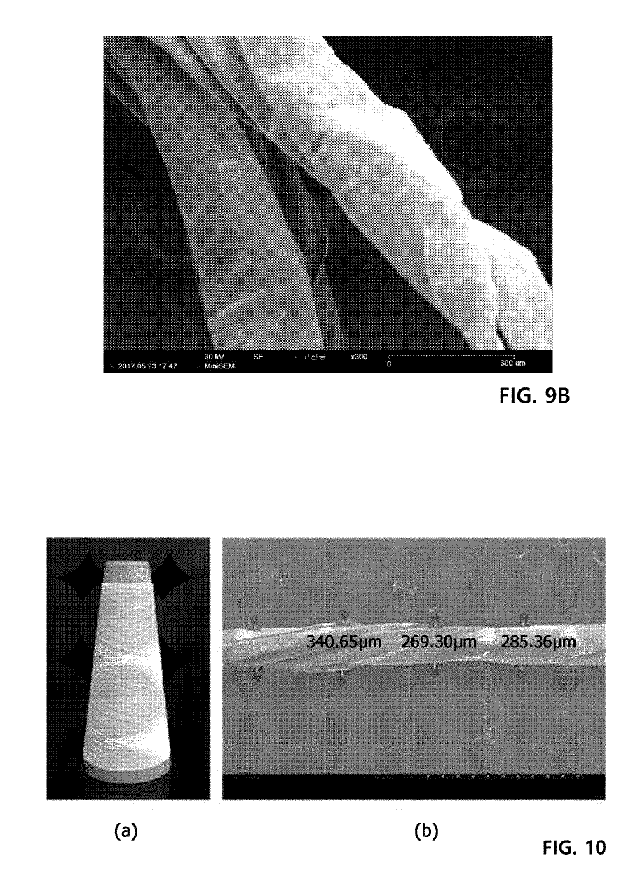

The roll of the prepared nanofiber web was first-slit to have a width of 5 mm as shown in FIG. 8A, and second-slit to have a width of 1.5 mm as shown in FIG. 8B, thereby obtaining slitting yarn, and an image of the slitting yarn produced in the second precision slitting process, which was wound, is shown in FIG. 8C. The produced slitting yarn, as shown in FIG. 3B, had a width of 1.5 mm. Two strands of the produced slitting yarn were Z-twisted to have a yarn twist number of 700T/M (twists/meter) using a 2-for-1 twisting machine as shown in FIG. 9A, and then untwisted in an opposite direction to have an untwist rate of 25%, represented by Mathematical Expression 1 below, thereby producing yarn for a cell scaffold as shown in FIG. 9B.

Untwist rate (%)=(length (m) of yarn after untwisting−length (m) of ply yarn)×100/length (m) of ply yarn [Mathematical Expression 1]

### COMPARATIVE EXAMPLE 1

Yarns for a cell scaffold as shown in FIGS. 10A and 10B were produced by the same method as described in Example 1, except that two strands of slitting yarn were Z-twisted to have a yarn twist number of 700T/M using a 2-for-1 twisting machine.

### COMPARATIVE EXAMPLE 2

Yarns for a cell scaffold as shown in FIGS. 10A and 10B were produced by the same method as described in Example 1, except that two strands of slitting yarn previously produced were not twisted.

### EXPERIMENTAL EXAMPLE

A plurality of strands of each of the yarns for a cell scaffold produced in Example 1 and Comparative Examples 1 and 2 were arranged parallel and fixed on a well plate for cell culture. Mesenchymal stem cells (MSCs) were loaded in the amount of 5×10⁴, 2.75×10⁵ or 2×10⁴ into the well plate including the yarn for a cell scaffold, and then proliferated in a DMEM+FBS or KBS-3 basal medium at 37° C. for 4 days.

Afterward, the cultured MSCs were stained with an AP or neutral red solution, incubated in an incubator for approximately 10 minutes, and then observed using an inverted microscope or incubated in an incubator for approximately 5 minutes after being treated with trypsin-EDTA, followed by calculation of a cell count using a blood counting chamber. For another method, cells were stained using a cell counting kit (CCK-8), and absorbance was measured using a UV-vis spectrometer. Here, a control was cells that were two-dimensionally cultured in a cell culture dish under the above-described culture conditions.

Among the absorbances measured according to Example 1 and Comparative Examples 1 and 2, provided that the absorbance of Example 1 is set as 100%, the absorbances of Comparative Examples 1 and 2 are relatively shown in Table 1 below.

A higher absorbance may be evaluated as cells that are well cultured after being settled in yarn for a cell scaffold.

TABLE 1

| | | Comparative | Comparative |
|---|---|---|---|
| | Example 1 | Example 1 | Example 2 |
| Relative absorbance (%) | 100 | 82 | 87 |

As shown in Table 1, it can be confirmed that, in the yarn for a cell scaffold according to Example 1, compared to those of Comparative Examples 1 and 2, settlement and culture of MSCs are well performed.

### Experimental Example 2

A plurality of strands of the yarn for a cell culture scaffold produced in Example 1 were arranged parallel and fixed on a well plate for cell culture. Fibroblasts (HS27) were loaded into the well plate including the yarn, and proliferated in a 10% complete medium at 37° C. for 2 days. Here, the 10% complete medium was prepared by mixing Ham's F12 medium with Dulbecco's Modified Eagle Medium (DMEM) at a volume ratio of 1:1.5, and adding 7 vol % of fetal bovine serum, 65 U/mL of penicillin and 65 μg/mL of streptomycin. Afterward, an SEM image of the proliferated fibroblasts was taken and is shown in FIG. 5, and a confocal microscope image of the fibroblasts taken after DAPI staining is shown in FIG. 6.

Referring to FIGS. 5 and 6, it can be confirmed that the fibroblasts are cultured in contact with an open space between the plurality of twisted fiber strands by partial untwisting, and it can be expected that, when fibroblasts are adhered onto different spaces shown in FIG. 5, the fibroblasts can be three-dimensionally cultured.

The following Table 2 shows amino acid sequences of SEQ ID NOS: 1 to 28 described in the present invention.

TABLE 2

| SEQ. | |
|---|---|
| ID. | |
| NO: | Amino acid sequence |
| 1 | Met Ala Lys Pro Ser Tyr Pro Pro Thr Tyr Lys Ala Lys Pro Ser |
| | Tyr Pro Pro Thr Tyr Lys Ala Lys Pro Ser Tyr Pro Pro Thr Tyr Lys |
| | Ala Lys Pro Ser Tyr Pro Pro Thr Tyr Lys Ala Lys Pro Ser Tyr Pro |
| | Pro Thr Tyr Lys Ala Lys Pro Ser Tyr Pro Pro Thr Tyr Lys Ser Ser |
| | Glu Glu Tyr Lys Gly Gly Tyr Tyr Pro Gly Asn Thr Tyr His Tyr His |
| | Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser |
| | Gly Lys Tyr Lys Tyr Leu Lys Lys Ala Arg Lys Tyr His Arg Lys Gly |
| | Tyr Lys Lys Tyr Tyr Gly Gly Ser Ser Ala Lys Pro Ser Tyr Pro Pro |
| | Thr Tyr Lys Ala Lys Pro Ser Tyr Pro Pro Thr Tyr Lys Ala Lys Pro |
| | Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr |
| | Pro Pro Thr Tyr Lys |
| 2 | Met Ala Lys Pro Ser Tyr Pro Pro Thr Tyr Lys Ala Lys Pro Ser |
| | Tyr Pro Pro Thr Tyr Lys Ala Lys Pro Ser Tyr Pro Pro Thr Tyr Lys |
| | Ala Lys Pro Ser Tyr Pro Pro Thr Tyr Lys Ala Lys Pro Ser Tyr Pro |
| | Pro Thr Tyr Lys Ala Lys Pro Ser Tyr Pro Pro Thr Tyr Lys Ser Ser |
| | Glu Glu Tyr Lys Gly Gly Tyr Tyr Pro Gly Asn Thr Tyr His Tyr His |
| | Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser |
| | Gly Lys Tyr Lys Tyr Leu Lys Lys Ala Arg Lys Tyr His Arg Lys Gly |
| | Tyr Lys Lys Tyr Tyr Gly Gly Ser Ser Ala Lys Pro Ser Tyr Pro Pro |
| | Thr Tyr Lys Ala Lys Pro Ser Tyr Pro Pro Thr Tyr Lys Ala Lys Pro |
| | Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr |
| | Pro Pro Thr Tyr Lys Gly Arg Gly Asp Ser Pro |
| 3 | Met Ala Lys Pro Ser Tyr Pro Pro Thr Tyr Lys Ala Lys Pro Ser |
| | Tyr Pro Pro Thr Tyr Lys Ala Lys Pro Ser Tyr Pro Pro Thr Tyr Lys |
| | Ala Lys Pro Ser Tyr Pro Pro Thr Tyr Lys Ala Lys Pro Ser Tyr Pro |
| | Pro Thr Tyr Lys Ala Lys Pro Ser Tyr Pro Pro Thr Tyr Lys Pro Trp |
| | Ala Asp Tyr Tyr Gly Pro Lys Tyr Gly Pro Pro Arg Arg Tyr Gly Gly |
| | Gly Asn Tyr Asn Arg Tyr Gly Arg Arg Tyr Gly Gly Tyr Lys Gly Trp |
| | Asn Asn Gly Trp Lys Arg Gly Arg Trp Gly Arg Lys Tyr Tyr Gly Ser |
| | Ala Lys Pro Ser Tyr Pro Pro Thr Tyr Lys Ala Lys Pro Ser Tyr Pro |
| | Pro Thr Tyr Lys Ala Lys Pro Ser Tyr Pro Pro Thr Tyr Lys Ala Lys |
| | Pro Ser Tyr Pro Pro Thr Tyr Lys Ala Lys Pro Ser Tyr Pro Pro Thr |
| | Tyr Lys Ala Lys Pro Ser Tyr Pro Pro Thr Tyr Lys Leu |
| 4 | Ala Asp Tyr Tyr Gly Pro Lys Tyr Gly Pro Pro Arg Arg Tyr Gly |
| | Gly Gly Asn Tyr Asn Arg Tyr Gly Arg Arg Tyr Gly Gly Tyr Lys Gly |
| | Trp Asn Asn Gly Trp Lys Arg Gly Arg Trp Gly Arg Lys Tyr Tyr |
| 5 | Ser Ser Glu Glu Tyr Lys Gly Gly Tyr Tyr Pro Gly Asn Thr Tyr |
| | His Tyr His Ser Gly Gly Ser Tyr His Gly Ser Gly Tyr His Gly Gly |
| | Tyr Lys Gly Lys Tyr Tyr Gly Lys Ala Lys Lys Tyr Tyr Tyr Lys Tyr |
| | Lys Asn Ser Gly Lys Tyr Lys Tyr Leu Lys Lys Ala Arg Lys Tyr His |
| | Arg Lys Gly Tyr Lys Lys Tyr Tyr Gly Gly Gly Ser Ser |
| 6 | Ala Lys Pro Ser Tyr Pro Pro Thr Tyr Lys |
| 7 | Ala Lys Pro Ser Tyr Pro Pro Thr Tyr Lys Ala Lys Pro Ser Tyr Pro |
| | Pro Thr Tyr Lys Ala Lys Pro Ser Tyr Pro Pro Thr Tyr Lys Ala Lys |
| | Pro Ser Tyr Pro Pro Thr Tyr Lys Ala Lys Pro Ser Tyr Pro Pro Thr |
| | Tyr Lys Ala Lys Pro Ser Tyr Pro Pro Thr Tyr Lys |
| 8 | Arg Gly Asp |
| 9 | Arg Gly Asp Ser |
| 10 | Arg Gly Asp Cys |
| 11 | Arg Gly Asp Val |
| 12 | Arg Gly Asp Ser Pro Ala Ser Ser Lys Pro |
| 13 | Gly Arg Gly Asp Ser |
| 14 | Gly Arg Gly Asp Thr Pro |
| 15 | Gly Arg Gly Asp Ser Pro |
| 16 | Gly Arg Gly Asp Ser Pro Cys |
| 17 | Tyr Arg Gly Asp Ser |
| 18 | Ser Pro Pro Arg Arg Ala Arg Val Thr |
| 19 | Trp Gln Pro Pro Arg Ala Arg Ile |
| 20 | Asn Arg Trp His Ser Ile Tyr Ile Thr Arg Phe Gly |
| 21 | Arg Lys Arg Leu Gln Val Gln Leu Ser Ile Arg Thr |
| 22 | Lys Ala Phe Asp Ile Thr Tyr Val Arg Leu Lys Phe |
| 23 | Ile Lys Val Ala Asn |
| 24 | Lys Lys Gln Arg Phe Arg His Arg Asn Arg Lys Gly Tyr Arg Ser |
| | Gln |
| 25 | Val Ala Glu Ile Asp Gly Ile Gly Leu |
| 26 | Pro His Ser Arg Asn Arg Gly Asp Ser Pro |
| 27 | Asn Arg Trp His Ser Ile Tyr Ile Thr Arg Phe Gly |
| 28 | Thr Trp Tyr Lys Ile Ala Phe Gln Arg Asn Arg Lys |

Embodiments of the present invention have been described above, but the spirit of the present invention is not limited to the embodiments presented herein, and it will be understood by those of ordinary skill in the art that other embodiments may be easily suggested by adding, changing, deleting or adding components within the scope of the same idea and they are also included in the scope of the spirit of the present invention.

1. Yarn for a cell culture scaffold, comprising:
a ply-twisted fiber strands; and
an open space between fibers by untwisting at least a part of the ply-twisted fiber strands to prevent density-dependent inhibition of cells to be cultured and increase a cell-contacting specific surface area.

2. The yarn according to claim 1, wherein the fiber is spun yarn, filament yarn or slitting yarn.

3. The yarn according to claim 1, wherein the fiber includes, as a fiber-forming component, any one or more non-biodegradable components selected from the group consisting of polystyrene (PS), polyethylene terephthalate (PET), polyethersulfone (PES), polyvinylidene fluoride (PVDF), polyacrylonitrile (PAN), polydimethylsiloxane (PDMS), a polyamide, a polyalkylene, a poly(alkylene oxide), a poly(amino acid), a poly(allylamine), polyphosphazene and a polyethyleneoxide-polypropyleneoxide block copolymer, or any one or more biodegradable components selected from the group consisting of polycaprolactone, polydioxanone, polyglycolic acid, poly(L-lactide) (PLLA), poly(DL-lactide-co-glycolide) (PLGA), polylactic acid and polyvinyl alcohol.

4. The yarn according to claim 1, wherein the yarn has a fineness of 20 to 300 deniers.

5. The yarn according to claim 1, wherein the fiber has a fineness of 0.1 to 30 deniers.

6. The yarn according to claim 2, wherein the slitting yarn is a fiber web having a three-dimensional network structure cut to have a predetermined width.

7. The yarn according to claim 6, wherein the fiber web has a basis weight of 0.1 to 100 g/m², and a width of 0.1 to 30 mm.

8. The yarn according to claim 1, wherein a physiologically active component inducing any one or more of adhesion, migration, growth, proliferation and differentiation of cells is further provided to the fiber.

9. The yarn according to claim 8, wherein the physiologically active component includes any one or more among any one or more compounds selected from the group consisting of a monoamine, an amino acid, a peptide, a saccharide, a lipid, a protein, a glucoprotein, a glucolipid, a proteoglycan, a mucopolysaccharide and a nucleic acid, and a cell.

10. The yarn according to claim 1, which is used in a scaffold to culture any one or more types of stem cells selected from the group consisting of totipotent stem cells, pluripotent stem cells, multipotent stem cells, oligopotent stem cells and single stem cells, and one or more types of differentiated cells selected from the group consisting of hematopoietic stem cells, liver cells, fiber cells, epithelial cells, mesothelial cells, endothelial cells, muscle cells, nerve cells, immune cells, adipose cells, cartilage cells, bone cells, blood cells and skin cells.

11. A fabric for a cell culture scaffold, comprising:
the yarn according to claim 1.

12. An implant for tissue engineering, comprising:
the fabric according to claim 11; and
cells cultured in contact with yarn for a cell culture scaffold included in the fabric.

13. The implant according to claim 12, wherein the cells are in contact with fibers spaced apart from each other in the yarn for a cell culture scaffold, and the fibers are arranged between adjacent cells among the cells to prevent intercellular contact.

14. The implant according to claim 12, wherein the cells include any one or more types of stem cells selected from the group consisting of totipotent stem cells, pluripotent stem cells, multipotent stem cells, oligopotent stem cells and single stem cells, and one or more types of differentiated cells selected from the group consisting of hematopoietic stem cells, liver cells, fiber cells, epithelial cells, mesothelial cells, endothelial cells, muscle cells, nerve cells, immune cells, adipose cells, cartilage cells, bone cells, blood cells and skin cells.


SEQUENCE LISTING (28 sequences)

SEQ ID NO: 1 - protein, 196 aa, Artificial Sequence
  Motif for cell culture scaffold
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYK

SEQ ID NO: 2 - protein, 202 aa, Artificial Sequence
  Motif for cell culture scaffold
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYKGRGDSP

SEQ ID NO: 3 - protein, 172 aa, Artificial Sequence
  Motif for cell culture scaffold
MAKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 4 - protein, 46 aa, Artificial Sequence
  Motif for cell culture scaffold
ADYYGPKYGPPRRYGGGNYNRYGRRYGGYKGWNNGWKRGRWGRKYY

SEQ ID NO: 5 - protein, 76 aa, Artificial Sequence
  Motif for cell culture scaffold
SSEEYKGGYYPGNTYHYHSGGSYHGSGYHGGYKGKYYGKAKKYYYKYKNSGKYKYLKKARKYHRKGYKKYYGGGSS

SEQ ID NO: 6 - protein, 10 aa, Artificial Sequence
  Motif for cell culture scaffold
AKPSYPPTYK

SEQ ID NO: 7 - protein, 60 aa, Artificial Sequence
  Motif for cell culture scaffold
AKPSYPPTYKAKPSYPPTYKAKPSYPPTYKAKPSYPPTYKAKPSYPPTYKAKPSYPPTYK

SEQ ID NO: 8 - protein, 3 aa, Artificial Sequence
  Motif for cell culture scaffold
RGD

SEQ ID NO: 9 - protein, 4 aa, Artificial Sequence
  Motif for cell culture scaffold
RGDS

SEQ ID NO: 10 - protein, 4 aa, Artificial Sequence
  Motif for cell culture scaffold
RGDC

SEQ ID NO: 11 - protein, 4 aa, Artificial Sequence
  Motif for cell culture scaffold
RGDV

SEQ ID NO: 12 - protein, 10 aa, Artificial Sequence
  Motif for cell culture scaffold
RGDSPASSKP

SEQ ID NO: 13 - protein, 5 aa, Artificial Sequence
  Motif for cell culture scaffold
GRGDS

SEQ ID NO: 14 - protein, 6 aa, Artificial Sequence
  Motif for cell culture scaffold
GRGDTP

SEQ ID NO: 15 - protein, 6 aa, Artificial Sequence
  Motif for cell culture scaffold
GRGDSP

SEQ ID NO: 16 - protein, 7 aa, Artificial Sequence
  Motif for cell culture scaffold
GRGDSPC

SEQ ID NO: 17 - protein, 5 aa, Artificial Sequence
  Motif for cell culture scaffold
YRGDS

SEQ ID NO: 18 - protein, 9 aa, Artificial Sequence
  Motif for cell culture scaffold
SPPRRARVT

SEQ ID NO: 19 - protein, 8 aa, Artificial Sequence
  Motif for cell culture scaffold
WQPPRARI

SEQ ID NO: 20 - protein, 12 aa, Artificial Sequence
  Motif for cell culture scaffold
NRWHSIYITRFG

SEQ ID NO: 21 - protein, 12 aa, Artificial Sequence
  Motif for cell culture scaffold
RKRLQVQLSIRT

SEQ ID NO: 22 - protein, 12 aa, Artificial Sequence
  Motif for cell culture scaffold
KAFDITYVRLKF

SEQ ID NO: 23 - protein, 5 aa, Artificial Sequence
  Motif for cell culture scaffold
IKVAN

SEQ ID NO: 24 - protein, 16 aa, Artificial Sequence
  Motif for cell culture scaffold
KKQRFRHRNRKGYRSQ

SEQ ID NO: 25 - protein, 9 aa, Artificial Sequence
  Motif for cell culture scaffold
VAEIDGIGL

SEQ ID NO: 26 - protein, 10 aa, Artificial Sequence
  Motif for cell culture scaffold
PHSRNRGDSP

SEQ ID NO: 27 - protein, 12 aa, Artificial Sequence
  Motif for cell culture scaffold
NRWHSIYITRFG

SEQ ID NO: 28 - protein, 12 aa, Artificial Sequence
  Motif for cell culture scaffold
TWYKIAFQRNRK